(19) United States
(12) Patent Application Publication
(10) Pub. No.: US 2005/0080125 A1
(43) Pub. Date: Apr. 14, 2005

(54) Novel medical uses of 4,5-dihydro-1H-pyrazole derivatives having CB1-antagonistic activity

(76) Inventors: Jochen Antel, Bad Munder (DE); Gunter Krause, Burgdorf (DE); Josephus Hubertus Lange, Almere (NL); Cornelis Gerrit Kruse, Amersfoort (NL); Peter-Colin Gregory, Hannover (GB); Cathaline Den Besten, Baambrugge (NL)

Correspondence Address: Finnegan, Henderson, Farabow,;Garrett & Dunner, L.L.P., 1300 I Street, N.W., Washington, DC, 20005-3315, US

(21) Appl. No.: 10/929,377

(22) Filed: Aug. 31, 2004

Related U.S. Application Data
(60) Provisional application No. 60/499,382, filed on Sep. 3, 2003.

(51) Int. Cl. A61K 31/415

(52) U.S. Cl. 514/406

(57) ABSTRACT

The present invention relates to a novel medical use of 4,5-dihydro-1H-pyrazole compounds which are potent antagonists of the cannabis CB₁-receptor. Said compounds are particularly suitable in the manufacture of medicaments for the treatment and/or prophylaxis of CB₁ receptor related diseases in juvenile patients and/or for the treatment and/or prophylaxis of drug induced obesity in juvenile as well as in adolescent patients. The compounds have the general formula (I) 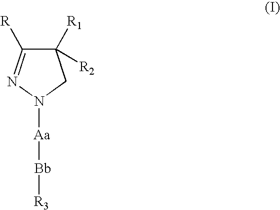
wherein the group Bb represents sulfonyl or carbonyl, and the substituents R, R₁, R₂ and R₃, and the group Aa are defined as shown in the description.

The present invention relates to a group of novel therapeutic and/or prophylactic uses of 4,5-dihydro-1H-pyrazole derivatives and to pharmaceutical compositions containing one or more of these compounds as an active component for the novel uses. The 4,5-dihydro-1H-pyrazoles are potent Cannabis-1 (CB₁) receptor antagonists with outstanding utility for the novel medical uses provided by the present invention.

Cannabinoids are present in the Indian hemp Cannabis Sativa L. and have been used as medicinal agents for centuries (Mechoulam, R.; Feigenbaum, J. J. Prog. Med. Chem. 1987, 24, 159). However, only within the past ten years the research in the cannabinoid area has revealed pivotal information on cannabinoid receptors and their (endogenous) agonists and antagonists. The discovery and the subsequent cloning of two different subtypes of Cannabinoid receptors (CB₁ and CB₂) stimulated the search for novel cannabinoid receptor antagonists (Munro, S.; Thomas, K. L.; Abu-Shaar, M. Nature 1993, 365, 61. Matsuda, L. A.; Bonner, T. I. Cannabinoid Receptors, Pertwee, R. G. Ed. 1995, 117, Academic Press, London). In addition, pharmaceutical companies became interested in the development of cannabinoid drugs for the treatment of diseases connected with disorders of the cannabinoid system. The wide distribution of CB₁ receptors in the brain, in combination with the strictly peripheral localisation of the CB₂ receptor, makes the CB₁ receptor a very interesting molecular target for CNS-directed drug discovery in the areas of both psychiatric and neurological disorders (Consroe, P. Neurobiology of Disease 1998, 5, 534. Pop, E. Curr. Opin. In CPNS Investigational Drugs 1999, 1, 587. Greenberg, D. A. Drug News Perspect. 1999, 12, 458). Hitherto, three types of distinct CB₁ receptor antagonists are known. Sanofi disclosed their diarylpyrazole congeners as selective CB₁ receptor antagonists. A representative example is. SR-141716A, which is currently undergoing Phase II clinical development for psychotic disorders (Dutta, A. K.; Sard, H.; Ryan, W.; Razdan, R. K.; Compton, D. R.; Martin, B. R. Med. Chem. Res. 1994, 5, 54. Lan, R.; Liu, Q.; Fan, P.; Lin, S.; Fernando, S. R.; McCallion, D.; Pertwee, R.; Makriyannis, A. J. Med. Chem. 1999, 42, 769. Nakamura-Palacios, E. M.; Moerschbaecher, J. M.; Barker, L. A. CNS Drug Rev. 1999, 5, 43). Aminoalkylindoles have been disclosed as CB₁ receptor antagonists. A representative example is Iodopravadoline (AM-630), which was introduced in 1995. AM-630 is a CB₁ receptor antagonist, but sometimes behaves as a weak partial agonist (Hosohata, K.; Quock, R. M.; Hosohata, Y.; Burkey, T. H.; Makriyannis, A.; Consroe, P.; Roeske, W. R.; Yamamura, H. I. Life Sc. 1997, 61, PL115). More recently, researchers from Eli Lilly described aryl-aroyl substituted benzofurans as selective CB₁ receptor antagonists (e.g. LY-320135) (Felder, C. C.; Joyce, K. E.; Briley, E. J.; Glass, M.; Mackie, K. P.; Fahey, K. J.; Cullinan, G. J.; Hunden, D. C.; Johnson, D. W.; Chaney, M. O.; Koppel, G. A.; Brownstein, M. J. Pharmacol. Exp. Ther. 1998, 284, 291). Recently, 3-alkyl-5,5′-diphenylimidazolidinediones were described as cannabinoid receptor ligands, which were indicated to be cannabinoid antagonists (Kanyonyo, M.; Govaerts, S. J.; Hermans, E.; Poupaert, J. H., Lambert, D. M. Biorg. Med. Chem. Lett. 1999, 9, 2233). Interestingly, many CB₁ receptor antagonists have been reported to behave as inverse agonists in vitro (Landsman, R. S.; Burkey, T. H.; Consroe, P.; Roeske, W. R.; Yamamura, H. I. Eur. J. Pharmacol. 1997, 334, R1). Recent reviews provide a nice overview of the current status in the cannabinoid research area (Mechoulam, R.; Hanus, L.; Fride, E. Prog. Med. Chem. 1998, 35, 199. Lambert, D. M. Curr. Med. Chem. 1999, 6, 635. Mechoulam, R.; Fride, E.; Di Marzo, V. Eur. J. Pharmacol. 1998, 359, 1).

From the international patent application WO 01/70700 4,5-dihydro-1H-pyrazole compounds are known which exhibit potent and selective cannabis CB₁-receptor antagonistic activity. These compounds have the formula (I) defined below, and have been suggested for use in the treatment of psychiatric disorders such as psychosis, anxiety, depression, attention deficits, memory disorders and appetite disorders, obesity, neurological disorders such as dementia, distonia, Parkinson's disease, Alzheimer's disease, epilepsy, Huntington's disease, Tourette's syndrome, cerebral ischaemia, as well as for the treatment of pain disorders and other CNS-diseases involving cannabinoid neurotransmission, and in the treatment of gastrointestinal disorders and cardiovascular disorders.

It has now surprisingly been found that the 4,5-dihydro-1H-pyrazole derivatives of the formula (I) which are potent and selective antagonists of the cannabis CB₁-receptor, prodrugs thereof, tautomers thereof and salts thereof 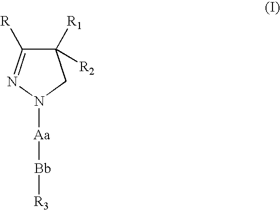
wherein
R and R₁ are the same or different and represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
Aa represents one of the groups (i), (ii), (iii), (iv) or (v) 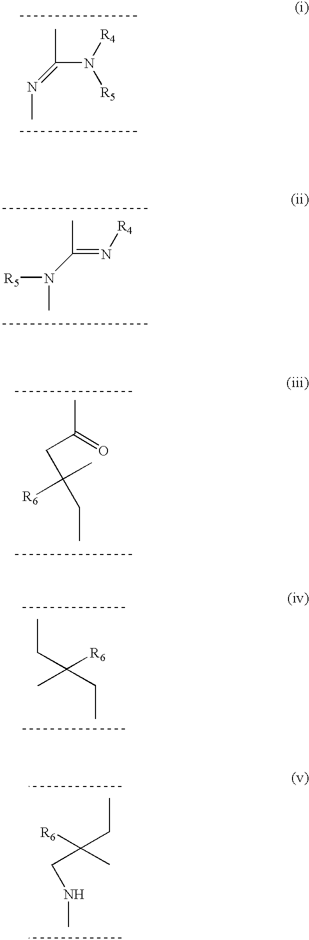
wherein
   R₄ and R₅ independently of each other represent hydrogen or C₁₋₈ branched or unbranched alkyl or C₃₋₈ cycloalkyl or R₄ represents acetamido or dimethylamino or 2,2,2-trifluoroethyl or phenyl or pyridyl with the proviso that R₅ represents hydrogen
   R₆ represents hydrogen or C₁₋₃ unbranched alkyl
Bb represents sulfonyl or carbonyl,
R₃ represents benzyl, phenyl, thienyl or pyridyl which may be substituted with 1, 2 or 3 substituents Y, which can be the same or different, or R₃ represents C₁₋₈ branched or unbranched alkyl or C₃₋₈ cycloalkyl, or R₃ represents naphtyl
   due to their unique pharmacological profile are particularly suited for the use in the manufacture of a medicaments for the treatment and/or prophylaxis of CB-1 receptor related diseases in juvenile patients and/or for the treatment and/or prophylaxis drug induced obesity in juvenile, as well as adolescent, patients. In this regard the compounds of formula (I) are highly valuable in providing medicaments for paediatric use on the one hand, and for the general use in drug induced obesity.

The outstanding unique pharmacological profile of compounds of formula (I) includes particularly high safety and tolerability which make the compounds particularly suitable in patient groups with enhanced need of safety and tolerability, in particular such as juvenile patients and/or patients subject to long term treatment, e.g. in drug induced obesity.

Due to the potent CB₁ antagonistic activity the compounds used according to the invention are suitable for use in the paediatric treatment and/or prophylaxis of psychiatric disorders such as psychosis, anxiety, depression, attention deficits, memory disorders and appetite disorders, obesity, neurological disorders such as dementia, distonia, Parkinson's disease, Alzheimer's disease, epilepsy, Huntington's disease, Tourette's syndrome, cerebral ischaemia, as well as for the treatment of pain disorders and other CNS-diseases involving cannabinoid neurotransmission, and in the treatment of gastrointestinal disorders and cardiovascular disorders, in young patients.

The affinity of the compounds of formula (I) for cannabinoid CB₁ receptors is described in the WO 01/70700, e.g. it was determined using membrane preparations of Chinese hamster ovary (CHO) cells in which the human cannabis CB₁ receptor is stably transfected in conjunction with [3H]CP-55,940 as radioligand. After incubation of a freshly prepared cell membrane preparation with the [3H]-ligand, with or without addition of compounds of the invention, separation of bound and free ligand was performed by filtration over glassfiber filters. Radioactivity on the filter was measured by liquid scintillation counting.

The cannabinoid CB₁ antagonistic activity of compounds of formula (I) is also described in the WO 01/70700, and was determined by functional studies using CHO cells in which human cannabinoid CB₁ receptors are stably expressed.

Adenylyl cyclase was stimulated using forskolin and measured by quantifying the amount of accumulated cyclic AMP. Concomitant activation of CB₁ receptors by CB₁ receptor agonists (e.g. CP-55,940 or (R)-WIN-55,212-2) can attenuate the forskolin-induced accumulation of cAMP in a concentration-dependent manner. This CB₁ receptor-mediated response can be antagonized by CB₁ receptor antagonists such as the compounds used in the present invention.

The whole content of the international patent application WO 01/70700 is incorporated by reference into the present application.

At least one centre of chirality is present (at the C₄ position of the 4,5-dihydro-1H-pyrazole moiety) in the compounds of the formula (I). The invention relates both to the novel use of racemates, mixtures of diastereomers and the individual stereoisomers of the compounds having formula (I). The invention also relates both to the novel use of the E isomer, Z isomer and E/Z mixtures of compounds having formula (I) wherein Aa has the meaning (i) or (ii) as described herein above.

According to one embodiment of the invention compound having formula (I) are used, wherein R is the group 4-chlorophenyl, R₁ is phenyl, R₂ is hydrogen, Aa is the group (i) wherein R₄ is hydrogen and R₅ is methyl, Bb is sulfonyl, and R₃ represents 4-chlorophenyl, and salts thereof. The compound having formula (I) used according to the invention may be a levorotatory enantiomer.

The compounds used in the present the invention can be obtained according to known methods. A suitable synthesis for the compounds used according to the present invention is described for compounds of formula (I) in the international patent application WO 01/70700. For example compounds having formula (III) (vide infra), wherein R₂ represents hydrogen can be also obtained according to methods known, for example: a) EP 0021506; b) DE 2529689.

Example compounds having been prepared according to WO 01/70700 and being investigated include the e.g. the following compounds:
1) 3-(4-Chlorophenyl)-4,5-dihydro-4-hydroxy-4-phenyl-1H-pyrazole
2) 3-(4-Chlorophenyl)-4,5-dihydro-N-((4-fluorophenyl)sulfonyl)-₄-phenyl-1H-pyrazole-1-carboxamidine
3) 4,5-Dihydro-N-((4-fluorophenyl)sulfonyl)-3-(4-methoxyphenyl)-4-(4-methoxy-phenyl)-1H-pyrazole-1-carboxamidine
4) 4,5-Dihydro-3-(4-methoxyphenyl)-4-(4-methoxyphenyl)-N-((4-methoxy-phenyl)sulfonyl)-1H-pyrazole-1-carboxamidine
5) 3-(4-Chlorophenyl)-4,5-dihydro-4-phenyl-N-((2,4,6-trimethylphenyl)sulfonyl)-1H-pyrazole-1-carboxamidine
6) 3-(4-Chlorophenyl)-4,5-dihydro-N-((4-fluorophenyl)sulfonyl)-4-hydroxy-4-phenyl-1H-pyrazole-1-carboxamidine
7) 3-(4-Chlorophenyl)-4,5-dihydro-N-(1-naphtoyl)-4-phenyl-1H-pyrazole-1-carboxamidine
8) 3-(4-Chlorophenyl)-4,5-dihydro-4-phenyl-N-(2-pyridoyl)-1H-pyrazole-1-carboxamidine
9) N¹,N¹-Dimethyl-N²-((4-chlorophenyl)sulfonyl)-3-(4-chlorophenyl)-4,5-dihydro-4-phenyl-1H-pyrazole-1-carboxamidine
10) N-Methyl-N′-((4-chlorophenyl)sulfonyl)-3-(4-chlorophenyl)-4,5-dihydro-4-(3-pyridyl)-1H-pyrazole-1-carboxamidine
11) N-Methyl-N′-((4-chlorophenyl)sulfonyl)-3-(4-chlorophenyl)-4,5-dihydro-4-(4-pyridyl)-1H-pyrazole-1-carboxamidine
12) N¹,N¹-Dimethyl-N²-((4-chlorophenyl)sulfonyl)-3-(4-chlorophenyl)-4,5-dihydro-4-hydroxy-4-phenyl-1H-pyrazole-1-carboxamidine
13) N-Ethyl-N′-((4-chlorophenyl)sulfonyl)-3-(4-chlorophenyl)-4,5-dihydro-4-hydroxy-4-phenyl-1H-pyrazole-1-carboxamidine
14) N-Methyl-N′-(3-(trifluoromethyl)benzoyl)-3-(4-chlorophenyl)-4,5-dihydro-4-phenyl-1H-pyrazole-1-carboxamidine
15) N-Methyl-N′-((4-chlorophenyl)sulfonyl)-3-(4-chlorophenyl)-4,5-dihydro-4-phenyl-1H-pyrazole-1-carboxamidine
16) N-Methyl-N′-((3-chlorophenyl)sulfonyl)-3-(4-chlorophenyl)-4,5-dihydro-4-phenyl-1H-pyrazole-1-carboxamidine
17) N-Methyl-N′-((4-chlorophenyl)sulfonyl)-3-(5-chloro-2-thienyl)-4,5-dihydro-4-phenyl-1H-pyrazole-1-carboxamidine
18) N-Propyl-N′-((4-fluorophenyl)sulfonyl)-3-(4-chlorophenyl)-4,5-dihydro-4-phenyl-1H-pyrazole-1-carboxamidine
19) N-(2-Propyl)-N′-((4-fluorophenyl)sulfonyl)-3-(4-chlorophenyl)-4,5-dihydro-4-phenyl-1H-pyrazole-1-carboxamidine
20) N-Methyl-N′-((2-propyl)sulfonyl)-3-(4-chlorophenyl)-4,5-dihydro-4-phenyl-1H-pyrazole-1-carboxamidine
21) N-(2-Propyl)-N′-((4-chlorophenyl)sulfonyl)-3-(4-pyridyl)-4,5-dihydro-4-phenyl-1H-pyrazole-1-carboxamidine
22) N¹-Ethyl-N¹-methyl-N²-((4-chlorophenyl)sulfonyl)-3-(4-chlorophenyl)-4,5-dihydro-4-phenyl-1H-pyrazole-1-carboxamidine
23) N¹-Ethyl-N¹-methyl-N²-((4-fluorophenyl)sulfonyl)-3-(4-chlorophenyl)-4,5-dihydro-4-phenyl-1H-pyrazole-1-carboxamidine
24) N¹,N¹-Dimethyl-N²-((4-(trifluoromethyl)phenyl)sulfonyl)-3-(4-chlorophenyl)-4,5-dihydro-4-phenyl-1H-pyrazole-1-carboxamidine
25) N¹,N¹-Dimethyl-N²-((3-methylphenyl)sulfonyl)-3-(4-chlorophenyl)-4,5-dihydro-4-phenyl-1H-pyrazole-1-carboxamidine
26) N¹,N¹-Dimethyl-N²-((3-methoxyphenyl)sulfonyl)-3-(4-chlorophenyl)-4,5-dihydro-4-phenyl-1H-pyrazole-1-carboxamidine
27) N-Ethyl-N′-((4-chlorophenyl)sulfonyl)-3-(4-chlorophenyl)-4,5-dihydro-4-phenyl-1H-pyrazole-1-carboxamidine
28) N-Dimethylamino-N′-((4-chlorophenyl)sulfonyl)-3-(4-chlorophenyl)-4,5-dihydro-4-phenyl-1H-pyrazole-1-carboxamidine
29) N-Methyl-N′-((4-(trifluoromethyl)phenyl)sulfonyl)-3-(4-chlorophenyl)-4,5-dihydro-4-phenyl-1H-pyrazole-1-carboxamidine
30) N¹,N¹-Dimethyl-N²-((2-methylphenyl)sulfonyl)-3-(4-chlorophenyl)-4,5-dihydro-4-phenyl-1H-pyrazole-1-carboxamidine
31) N-Methyl-N′-((2,4-difluorophenyl)sulfonyl)-3-(4-chlorophenyl)-4,5-dihydro-4-phenyl-1H-pyrazole-1-carboxamidine
32) N-Acetamido-N′-((4-chlorophenyl)sulfonyl)-3-(4-chlorophenyl)-4,5-dihydro-4-phenyl-1H-pyrazole-1-carboxamidine
33) N-(2,2,2-Trifluoroethyl)-N′-((4-chlorophenyl)sulfonyl)-3-(4-chlorophenyl)-4,5-dihydro-4-phenyl-1H-pyrazole-1-carboxamidine
34) N-(2-Pyridyl)-N′-((4-chlorophenyl)sulfonyl)-3-(4-chlorophenyl)-4,5-dihydro-4-phenyl-1H-pyrazole-1-carboxamidine
35) N-(4-Pyridyl)-N′-((4-chlorophenyl)sulfonyl)-3-(4-chlorophenyl)-4,5-dihydro-4-phenyl-1H-pyrazole-1-carboxamidine
36) N-Phenyl-N′-((4-chlorophenyl)sulfonyl)-3-(4-chlorophenyl)-4,5-dihydro-4-phenyl-1H-pyrazole-1-carboxamidine
37) 3-(4-Chlorophenyl)-1-[3-((4-chlorophenyl)sulfonyl)butanoyl]-4,5-dihydro-4-phenyl-1H-pyrazole
38) 3-(4-Chlorophenyl)-1-[3-(phenylsulfonyl)propanoyl]-4,5-dihydro-4-phenyl-1H-pyrazole
39) 3-(4-Chlorophenyl)-1-[3-((4-chlorophenyl)sulfonyl)propanoyl]-4,5-dihydro-4-phenyl-1H-pyrazole
40) 3-(4-Chlorophenyl)-4,5-dihydro-4-phenyl-1-[2-((3-(trifluoromethyl)phenyl)sulfonyl)ethyl]-1H-pyrazole
41) 3-(4-Chlorophenyl)-1-[2-(benzylsulfonyl)ethyl]-4,5-dihydro-4-phenyl-1H-pyrazole
42) 3-(4-Chlorophenyl)-1-[2-((4-chlorophenyl)sulfonyl)ethyl]4,5-dihydro-4-phenyl-1H-pyrazole
43) 3-(4-Chlorophenyl)-1-[2-((4-chlorophenyl)sulfonyl)ethyl]4,5-dihydro-4-hydroxy-4-phenyl-1H-pyrazole
44) N-[2-(3-(4-Chlorophenyl)-4,5-dihydro-4-phenyl-1H-pyrazol-1-yl)ethyl]-3-(trifluoromethyl)benzenesulfonamide.

The compounds used according to the invention can be brought into forms suitable for paediatric administration, as well as for the administration in treating drug induced obesity by means of usual processes using auxiliary substances and/or liquid or solid carrier materials.

Hence, in a further aspect the invention also pertains to a pharmaceutical composition containing at least one compound of formula (I) as an active component for the treatment and/or prophylaxis of CB₁ receptor related diseases in juvenile patients and/or for the treatment and/or prophylaxis of drug induced obesity in juvenile as well as adolescent patients, and at least one auxiliary excipient. In such a pharmaceutical composition the compound of formula (I) is preferably present in an amount effectively suited for the treatment and/or prophylaxis of a psychiatric disorder, a gastrointestinal disorder, a cardiovascular disorder, or a combination of said disorders, in a juvenile patient in need of such treating.

In a further embodiment of the invention in the pharmaceutical composition the compound of formula (I) is present in an amount effectively suited for the treatment and/or prophylaxis of drug induced obesity in juvenile as well as adolescent patients in need of such treating.

Finally the invention also includes a method of treatment and/or prophylaxis of CB₁ receptor related diseases in juvenile patients and/or for the treatment and/or prophylaxis of drug induced obesity in juvenile as well as adolescent patients, characterized in that a compound of formula (I) is administered to said patient in need of such treating. The method of treatment and/or prophylaxis according to the invention may be further characterized in that the treating is directed to psychiatric disorders such as psychosis, anxiety, depression, attention deficits, memory disorders and appetite disorders, obesity, including drug induced obesity, neurological disorders such as Parkinson's disease, dementia, distonia, Alzheimer's disease, epilepsy, Huntington's disease, Tourette's syndrome, ischemia, pain and other CNS-diseases involving cannabinoid neurotransmission. Preferably, in one embodiment of the invention the method of treatment and/or prophylaxis is directed to the treating of obesity in juvenile patients. In another preferred embodiment of the invention the method of treatment and/or prophylaxis is directed to the treating of drug induced obesity in juvenile or adolescent patients. This drug induced obesity may be in particular caused by drugs like atypical antipsychotics.

In one embodiment of the invention the method of treatment and/or prophylaxis is directed to the treating of obesity in juvenile patients. Thus, it is advantageous that Cannabinoid antagonists are suitable for the treatment of Childhood Obesity and related Comorbidities as for example Type 2 Diabetes. There is a clear medical need for improved therapy as obesity has become an increasingly important medical problem not only in the adult population but increasingly in children and (young and older) adolescents. In national surveys from the 1960s to the 1990s in the United States, the prevalence of overweight in children grew from 5% to 11% (Sorof and Daniels 2002). In Canada as another example childhood obesity has tripled in the past 20 years (Spurgeon 2002). Obesity in childhood causes a wide range of serious complications, and increases the risk of premature illness and death later in life, raising public-health concerns (Ebbeling, Pawlak et al. 2002). Over the last decades a tremendous increase of cases of type 2 diabetes was observed, especially also in children. This epidemic trend is clearly reflecting the increasing rates of obesity. Type-2-diabetes was in the past considered a disease of adults and older individuals, not a paediatric condition (Arslanian 2002). One of the main risk factor of paediatric type 2 diabetes is obesity.

Type 2 diabetes in children (as is in adults) is part of the insulin resistance syndrome (Rosenbloom 2002) that includes hypertension, dyslipidemia and other atherosclerosis risk factors, and hyperandrogenism seen as premature adrenarche and polycystic ovary syndrome. Other outcomes related to childhood obesity include left ventricular hypertrophy, nonalcoholic steatohepatitis, obstructive sleep apnea, orthopedic problems, and severe psychosocial problems.

In addition primary hypertension has become increasingly common in children again associated obesity as a major independent risk factor. Obese children are at approximately a 3-fold higher risk for hypertension than non-obese children (Sorof and Daniels 2002). The benefits of weight loss for blood pressure reduction in children have been demonstrated in both observational and interventional studies.

Public concerns are rising because of a rapid development of the childhood obesity epidemic in genetically stable populations. Driving factors are assumed to be mainly adverse environmental factors for which straightforward recommendations of life style modifications exists. Obesity and it's related co-morbidities are very serious medical conditions and state of the art measures and treatment of obesity and especially childhood obesity remain largely ineffective at the time being (Ebbeling, Pawlak et al. 2002). The management of type 2 diabetes in is also especially difficult in children and the adolescent age group (Silink 2002). Craving for and over consumption of palatable food is one of the important factors of life-style related obesity in humans and especially also in children and adolescents. Treatment of type 2 diabetes and other co-morbid conditions by the degree of metabolic derangement and symptoms: The only data on the use of oral hypoglycemic agents in children with type 2 diabetes has been with metformin (Rosenbloom 2002).

Thus, CB₁ antagonists used according to the present invention offer a unique opportunity for the treatment of obesity by interacting with these “driving forces”. They are superior to current medical treatments and especially suited for pediatric treatment because of their outstanding safety profile and/or tolerability. Treatment of obesity especially childhood obesity is besides efficacy dictated by safety.

Obesity in childhood is a medical condition that is likely to require long-term management. The safety profile of CB₁ antagonists according to the present invention are suggested to be superior to current standard medications, and these CB₁ antagonists will be especially suited for the treatment and prevention of childhood obesity and related co-morbidities.

### LITERATURE

Arslanian, S. (2002). “Type 2 diabetes in children: clinical aspects and risk factors.” Horm Res 57 Suppl 1: 19-28.
Ebbeling, C. B., D. B. Pawlak, et al. (2002). “Childhood obesity: public-health crisis, common sense cure.” Lancet 360(9331): 473-82.
Rosenbloom, A. L. (2002). “Increasing incidence of type 2 diabetes in children and adolescents: treatment considerations.” Paediatr Drugs 4(4): 209-21.
Silink, M. (2002). “Childhood diabetes: a global perspective.” Horm Res 57 Suppl 1: 1-5.
Sorof, J. and S. Daniels (2002). “Obesity hypertension in children: a problem of epidemic proportions.” Hypertension 40(4): 441-7.
Spurgeon, D. (2002). “Childhood obesity in Canada has tripled in past 20 years.” Bmj 324(7351): 1416.

In another embodiment of the invention the method of treatment and/or prophylaxis is directed to the treating of drug induced obesity in juvenile or adolescent patients. Drug induced weight gain is also of major concern and subject to high medical need of improved treatments. Again, in this context the CB₁ antagonists according to the present invention are suggested to be superior to current standard medications, and these CB₁ antagonists will be especially suited for the treatment and prevention of drug induced obesity in juvenile as well as in adolescent patients.

Regarding drug induced weight gain, it is reported by Zimmermann, U., T. Kraus, et al. (2003, “Epidemiology, implications and mechanisms underlying drug-induced weight gain in psychiatric patients.” J Psychiatr Res 37(3): 193-220) that body weight gain frequently occurs during drug treatment of psychiatric disorders and is often accompanied by increased appetite or food craving. While occurrence and time course of this side effect are difficult to predict, it ultimately results in obesity and the morbidity associated therewith in a substantial part of patients, often causing them to discontinue treatment even if it is effective. Weight gain appears to be most prominent in patients treated with some of the second generation antipsychotic drugs and with some mood stabilizers. Marked weight gain also frequently occurs during treatment with most tricyclic antidepressants.

Very large weight gains are associated with drugs like for example the atypical antipsychotics clozapine and olanzapine. Some atypical antipsychotics, however, tend to cause significant weight gain, which may lead to poor compliance and other adverse health effects (Nasrallah, H. (2003). “A review of the effect of atypical antipsychotics on weight.” Psychoneuroendocrinology 28 Suppl 1: 83-96.). The mechanisms involved in antipsychotic drug-related weight gain are as yet uncertain, although serotoninergic, histaminic, and adrenergic affinities have been implicated along with other metabolic mechanisms. The atypical antipsychotics vary in their propensity to cause weight change with long-term treatment. Follow-up studies show that the largest weight gains are associated with clozapine and olanzapine, and the smallest with quetiapine and ziprasidone. Risperidone is associated with modest weight changes that are not dose related. Given the equivalent efficacy of atypical antipsychotics, weight-gain profile is a legitimate factor to consider when constructing an algorithm for treatment due to the serious medical consequences of obesity. In this regard co-administration of CB₁ antagonist according to the invention is suggested to work beneficially.

1. Use of a CB₁ receptor antagonistic compound of formula (I), prodrugs thereof, tautomers thereof and salts thereof, in the manufacture of medicaments for the treatment and/or prophylaxis of CB₁ receptor related diseases in juvenile patients and/or for the treatment and/or prophylaxis of drug induced obesity in juvenile as well as in adolescent patients: 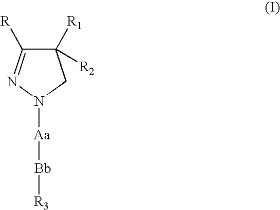
wherein
R and R₁ are the same or different and represent phenyl, thienyl or pyridyl which groups may be substituted with 1, 2 or 3 substituents Y, which can be the same or different, from the group C₁₋₃-alkyl or alkoxy, hydroxy, halogen, trifluoromethyl, trifluoromethylthio, trifluoromethoxy, nitro, amino, mono- or dialkyl (C₁₋₂)-amino, mono- or dialkyl (C₁₋₂)-amido, (C₁₋₃)-alkyl sulfonyl, dimethylsulfamido, C₁₋₃-alkoxycarbonyl, carboxyl, trifluoromethylsulfonyl, cyano, carbamoyl, sulfamoyl and acetyl, or R and/or R₁ represent naphtyl,
R₂ represents hydrogen, hydroxy, C₁₋₃-alkoxy, acetyloxy or propionyloxy,
Aa represents one of the groups (i), (ii), (iii), (iv) or (v) 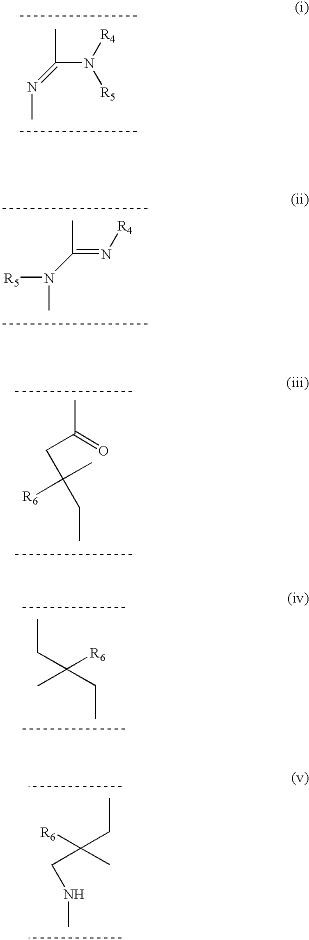
wherein
R₄ and R₅ independently of each other represent hydrogen or C₁₋₈ branched or unbranched alkyl or C₃₋₈ cycloalkyl or R₄ represents acetamido or dimethylamino or 2,2,2-trifluoroethyl or phenyl or pyridyl with the proviso that R₅ represents hydrogen
R₆ represents hydrogen or C₁₋₃ unbranched alkyl
Bb represents sulfonyl or carbonyl,
R₃ represents benzyl, phenyl, thienyl or pyridyl which may be substituted with 1, 2 or 3 substituents Y, which can be the same or different, or R₃ represents C₁₋₈ branched or unbranched alkyl or C₃₋₈ cycloalkyl, or R₃ represents naphtyl.

2. Use of the compound having formula (I) according to claim 1, wherein R is the group 4-chlorophenyl, R₁ is phenyl, R₂ is hydrogen, Aa is the group (i) wherein R₄ is hydrogen and R₅ is methyl, Bb is sulfonyl, and R₃ represents 4-chlorophenyl, and salts thereof.

3. Use of the compound having formula (I) according to claim 1, wherein the compound is a levorotatory enantiomer.

4. A pharmaceutical composition containing at least one compound of formula (I) as defined in claim 1 as an active component for the treatment and/or prophylaxis of CB₁ receptor related diseases in juvenile patients and/or for the treatment and/or prophylaxis of drug induced obesity in juvenile as well as adolescent patients, and at least one auxiliary excipient.

5. A pharmaceutical composition according to claim 4, wherein the at least one compound of formula (I) is present in an amount effectively suited for the treatment and/or prophylaxis of a psychiatric disorder, a gastrointestinal disorder, a cardiovascular disorder, or a combination of said disorders, in a juvenile patient in need of such treating.

6. A pharmaceutical composition according to claim 4, wherein the at least one compound of formula (I) is present in an amount effectively suited for the treatment and/or prophylaxis of drug induced obesity in juvenile as well as adolescent patients in need of such treating.

7. A method of treatment and/or prophylaxis of CB₁ receptor related diseases in juvenile patients and/or for the treatment and/or prophylaxis of drug induced obesity in juvenile as well as adolescent patients, characterized in that a compound of formula (I) as defined in claim 1 is administered to said patient in need of such treating.

8. A method of treatment and/or prophylaxis according to claim 7, characterized in that the treating is directed to psychiatric disorders such as psychosis, anxiety, depression, attention deficits, memory disorders and appetite disorders, obesity, including drug induced obesity, neurological disorders such as Parkinson's disease, dementia, distonia, Alzheimer's disease, epilepsy, Huntington's disease, Tourette's syndrome, ischaemia, pain and other CNS-diseases involving cannabinoid neurotransmission.

9. A method of treatment and/or prophylaxis according to claim 8, characterized in that the treating is directed to obesity in juvenile patients.

10. A method of treatment and/or prophylaxis according to claim 8, characterized in that the treating is directed to drug induced obesity in juvenile or adolescent patients.
